(19) United States
(12) Patent Application Publication
(10) Pub. No.: US 2014/0314867 A1
(43) Pub. Date: Oct. 23, 2014

(54) VITAMIN K2 MICROSPHERES

(71) Applicant: Medical and Pharmaceutical Industry Technology and Development Center, New Taipei City (TW)

(72) Inventors: Shu-Wen Whu, Yangmei City (TW); Chung-Chih Cheng, Taipei City (TW); Chih-Chiang Yang, Tainan City (TW)

(73) Assignee: Medical and Pharmaceutical Industry Technology and Development Center, New Taipei City (TW)

(21) Appl. No.: 14/258,860

(22) Filed: Apr. 22, 2014

Related U.S. Application Data
(60) Provisional application No. 61/814,801, filed on Apr. 22, 2013.

(51) Int. Cl. A61K 9/16; A61K 31/122

(52) U.S. Cl. CPC A61K 9/1647; A61K 31/122
USPC 424/501; 514/681

(57) ABSTRACT

A vitamin K₂ microsphere. The microsphere includes a poly(lactide-co-glycolide) acid (PLGA) particle, in which the Mw of PLGA is between 1000 and 300000, and the molar ratio between the lactide repeat units and the glycolide repeate unit is 1-9:9-1; and one or more vitamin K₂ molecules are embedded in the PLGA particle, wherein the vitamin K₂ is present in an amount of 0.005-75 wt %, based on the weight of the microsphere. Also disclosed are a method of preparing the vitamin K₂ microsphere, a method of treating osteoporosis using this microsphere, and a pharmaceutical composition containing the microsphere.

### CROSS REFERENCE TO RELATED APPLICATIONS

The application claims priority to both U.S. Provisional Application No. 61/814,801, filed on Apr. 22, 2013 and a subsequently filed Taiwanese patent application entitled “Vitamin K₂ microsphere, manufacture method, use, and drug thereof”, the contents of which are hereby incorporated by reference in their entirety.

### BACKGROUND OF THE INVENTION

Osteonecrosis, i.e., avascular necrosis of bone, is the death of bone cells caused by decreased blood flow to these cells. According to statistics, there are approximately 300,000 to 600,000 cases that occur in the US every year, and it usually affects people between 20 and 50 years of age.

In recent years, many studies have pointed out that vitamin K₂ can effectively inhibit osteoclast activity, which disassembles bone tissues, and promote bone regeneration. Further; vitamin K₂ can induce osteoblast cells, which synthesize bone, to differentiate bone cells at the osteonecrosis site from those at normal sites, and help repair the injured bone tissue (e.g., necrosis). It can also avoid the disadvantage of the hard-to-control growth factor activity. Therefore, vitamin K₂ is useful in treating osteoporosis.

However, due to conventional in vivo delivery routes (e.g., oral), the efficacy of vitamin K₂ can be maintained only for a short time as it is metabolized very fast in the body, requiring inconvenient multiple dosages in a day. Moreover, to achieve therapeutic effects, vitamin K₂ is taken at a high dosage, causing various side effects.

There is a need to develop a system that can release vitamin K₂ in an extended, controllable manner and at a low dose.

### BRIEF SUMMARY OF THE INVENTION

The invention is based on an unexpected discovery of a vitamin K₂ microsphere capable of releasing vitamin K₂ in a controlled manner.

One aspect of this invention relates to a vitamin K₂ microsphere that contains a particle formed of a poly(lactic-co-glycolic acid) (PLGA) and vitamin K₂. The PLGA, the viscosity of which can be 0.1-3 dl/g, has a molecular weight of 1000-300000 and contains lactic acid repeat units and glycolic acid repeat units. The molar ratio between the lactic acid repeat units and the glycolic acid repeat units is 1-9:9-1. Vitamin K₂ is embedded in the particle and constitutes 0.005-75% by weight of the vitamin K₂ microsphere.

The vitamin K₂ microsphere can have a particle size of 1-150 μm and contains vitamin K₂ in the amount of 0.01-0.3 mg.

Another aspect of this invention relates to a method of preparing the vitamin K₂ microsphere described above. The method includes the steps of: (a) providing a vitamin K₂ solution that contains vitamin K₂, the PLGA mentioned above, and a first solvent; (b) providing a polyvinyl alcohol (PVA) solution that contains polyvinyl alcohol and a second solvent; (c) forming a vitamin K₂ emulsion by mixing the vitamin K₂ solution with the PVA solution; and (d) removing the first and second solvents to obtain vitamin K₂ microspheres, each of which contains the PLGA and vitamin K₂ embedded in a particle formed of the PLGA.

The first solvent is an organic solvent, e.g., dichloromethane, chloroform, tetrahydrofuran, dimethylformamide, benzene, toluene, or a combination thereof. The weight/volume ratio between vitamin K₂ and the first solvent is 0.005-75%. The second solvent is water.

The vitamin K₂ microspheres thus obtained can be purified via filtration or centrifugation.

Optionally, a plasticizer is added at step (c) to mix with the vitamin K₂ solution and the PVA solution to form the vitamin K₂ emulsion.

Also within the scope of this invention is a method of treating osteoporosis by administering to a subject in need thereof an effective amount of the above-described vitamin K₂ microsphere.

Still within the scope of this invention is a pharmaceutical composition containing this vitamin K₂ microsphere and a pharmaceutically acceptable carrier.

This invention further includes use of the vitamin K₂ microsphere in the manufacture of a medicament for treating osteoporosis or for repairing damaged bone tissues.

The details of one or more embodiments of the invention are set forth in the description below. Other features, objects, and advantages of the invention will be apparent from the description and the figures, and from the claims.

### BRIEF DESCRIPTION OF THE DRAWINGS

FIG. 1 is a schematic cross-section of a vitamin K₂ microsphere (VK₂MS) of an embodiment of the present disclosure.

FIG. 2 shows a flow chart of a method of manufacturing the vitamin K₂ microsphere (VK₂MS) according to embodiments of the present disclosure.

FIGS. 3A-3D show surface morphology of the vitamin K₂ microsphere (VK₂MS) with different concentrations under a scanning electron microscope (SEM).

FIG. 4A shows an absorption spectrum of attenuated total reflectance-fourier transform infrared spectroscopy (ATR-FTIR) of the vitamin K₂ microsphere (VK₂MS) according to an embodiment of the present disclosure.

FIG. 4B shows an absorption spectrum of attenuated total reflectance-fourier transform infrared spectroscopy (ATR-FTIR) of the vitamin K₂ microsphere (VK₂MS) after UV irradiation according to an embodiment of the present disclosure.

FIG. 5 shows a particle size distribution of the vitamin K₂ microsphere (VK₂MS) according to an embodiment of the present disclosure.

FIG. 6 shows the remaining amount of different concentrations of vitamin K₂ microsphere (VK₂MS) after degradation experiments.

FIG. 7A shows the in vitro cumulative release amount of different concentrations of vitamin K₂ microsphere (VK₂MS).

FIG. 7B shows the in vitro cumulative release percentage of different concentrations of vitamin K₂ microsphere (VK₂MS).

FIG. 8 is a schematic diagram of the release of vitamin K₂ by vitamin K₂ microsphere (VK₂MS).

FIG. 9A shows the cell number after co-culturing different concentrations of vitamin K₂ and MG-63 cells for 1, 3, and 7 days.

FIG. 9B shows the cell number after co-culturing different concentrations of vitamin K₂ microsphere (VK₂MS) and MG-63 cells for 1, 3, and 7 days.

FIG. 10A shows the results of an alkaline phosphatase activity analysis after co-culturing different concentrations of vitamin K₂ and MG-63 cells for 1, 3, and 7 days.

FIG. 10B shows the results of an alkaline phosphatase activity analysis after co-culturing different concentrations of vitamin K₂ microsphere (VK₂MS) and MG-63 cells for 1, 3, and 7 days.

FIG. 11A shows the results of alkaline phosphatase activity analysis of a single cell after co-culturing different concentrations of vitamin K₂ and MG-63 cells for 1, 3, and 7 days.

FIG. 11B shows the results of alkaline phosphatase activity analysis of a single cell after co-culturing different concentrations of vitamin K₂ microsphere (VK₂MS) and MG-63 cells for 1, 3, and 7 days.

### DETAILED DESCRIPTION OF THE INVENTION

The vitamin K₂ microsphere of this invention contains a particle formed of PLGA, a biodegradable polymer, and vitamin K₂ that is embedded in the particle. As the polymer degrades, vitamin K₂ is slowly released in a controlled manner. The vitamin K₂ microsphere is useful for bone tissue regeneration.

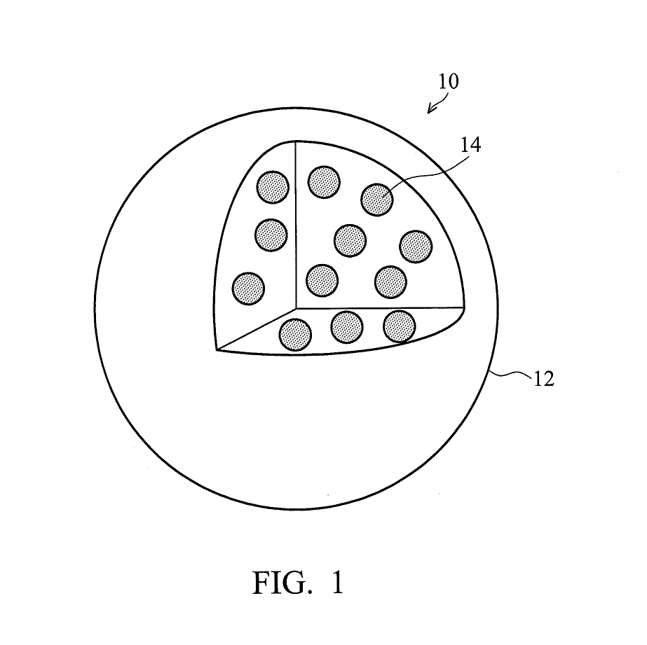

FIG. 1 is a schematic cross-section of a vitamin K₂ microsphere (VK₂MS) 10 of an embodiment of the present disclosure. The vitamin K₂ microsphere (VK₂MS) includes poly(lactide-co-glycolide) acid (PLGA) particle 12, which has a Mw of 1000-300000 (e.g., 4000-15000) and contains lactic acid repeat units and glycolic acid repeat units (the molar ratio is 1-9: 9-1, e.g., 3:1); and vitamin K₂ 14 embedded in the PLGA particle 12, wherein vitamin K₂ is present in an amount of 0.005-75 wt % based on the weight of the microsphere.

The particle size distribution of the vitamin K₂ microsphere can be between 1 μm and 150 μm, for example, between 2 μm and 100 μm. The particle size of the vitamin K₂ microsphere varies depending on the amount of encapsulated vitamin K₂. Generally, the particle size increases when the concentration of vitamin K₂ increases. The microsphere slowly releases vitamin K₂ into the bone cell growth environment to promote bone formation and help bone tissue reconstruction. Accordingly, to achieve delayed release, it is preferred that the particle size of the microsphere be controlled in an appropriate range. When the particle size is too small, microspheres may not stay at a target site. When the particle size is too large, controlled drug release may not be achieved. As such, the particle size of the microsphere is critical. The vitamin K₂ microsphere of this invention can be prepared by a nonaqueous phase separation method. Known nonaqueous phase separation methods include non-solvent phase precipitation, temperature dropping, solvent distillation, and a combination thereof. See Gast et al., J. of Colloid and Interface Science 1983, 96, 251-67. Non-solvent phase precipitation and solvent distillation can be used in combination to prepare the vitamin K₂ microsphere.

The vitamin K₂ microsphere can contain vitamin K₂ in the amount of 0.01-0.3 mg. The viscosity of the PLGA can be 0.1-3 dl/g (e.g., 0.14-0.22 dl/g).

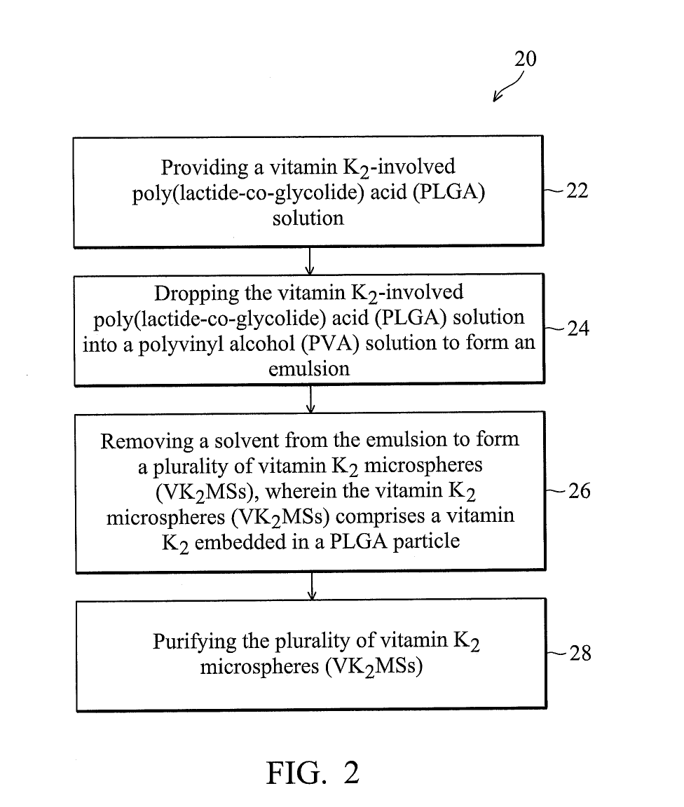

FIG. 2 shows flow chart 20 of a method for preparing the vitamin K₂ microsphere of this invention, which has pores on its surface. In step 22, a vitamin K₂ organic solution is provided, which contains vitamin K₂, the PLGA, and an organic solvent. The size of pores on the surface pore of the vitamin K₂ microsphere can be controlled by using different solvent-nonsolvent systems or different solvent evaporation rates. In one example, vitamin K₂ is encapsulated in a PLGA particle, the pore size of which is determined by using the dichloromethane-polyvinyl alcohol (PVA) system. Other than dichloromethane, chloroform, tetrahydrofuran, dimethyl formamide, benzene, and toluene can also be used.

More specifically, vitamin K₂ (e.g., 0.001-0.1 g) and PLGA (e.g., 0.0013-200 g) are dissolved in a solvent (e.g., dichloromethane) by stirring in an ice bath. The weight/volume ratio of the vitamin K₂ and PLGA can be 0.005-75% (e.g., 0.01-1%).

Subsequently, in step 24 shown in FIG. 2, the vitamin K₂ solution is added in a dropwise manner to a PVA aqueous solution to form an emulsion. The concentration of PVA can be 0.05-20 wt %. The PVA solution is optionally cooled in an ice bath before mixing with the vitamin K₂ solution. The concentration of vitamin K₂ in PLGA has a significant impact on its release rate. As such, in this emulsion-forming step, a plasticizer can be added to change the crosslinking density or modify the material so that vitamin K₂ is evenly dispersed in PLGA to achieve a controllable release rate. Examples of the plasticizer include sebacates, adipates, terephthalates, dibenzoates, gluterates, phthalates, azelates, nitrile, polychloroprene, EPDM, chlorinated polyethylene, and epichlorohydrin.

Next, in step 26 shown in FIG. 2, the organic solvent is removed from the emulsion to form a plurality of vitamin K₂ microspheres, each of which includes vitamin K₂ embedded in a PLGA particle. In this step, while the solvents are removed, PLGA is precipitated out and self-ensemble into particles, which encapsulate vitamin K₂ to form microspheres. Any suitable method, such as evaporation by stirring, heating, decompression, or a combination thereof, can be used to remove the solvents from the emulsion.

In Step 28 shown in FIG. 2, vitamin K₂ microspheres thus obtained are then purified. First, larger microspheres are filtered by a cell sieve to obtain a filtrate containing uniformly dispersed microspheres. Larger microspheres can cause aggregation and interfere with stable controllable release of vitamin K₂. A filtration step is thus performed to remove these larger microspheres. Subsequently, the filtrate is centrifuged. Note that the microspheres thus prepared are mixed with PVA. To remove PVA, the filtrate is diluted with water and centrifuged. The aliquot is removed. This water-washing is repeated several times. Subsequently, the centrifuged solution is rapidly cooled using liquid nitrogen, followed removal of water by being freeze-dried to obtain dried vitamin K₂ microspheres, which are stored in a drying cabinet for direct use or for preparing a pharmaceutical composition.

Not to be bound by any of the theory, PLGA can encapsulate different amounts of vitamin K₂, which is then released in a controllable manner into a target site at a desired concentration. By encapsulating different amounts of vitamin K₂, the size of the microspheres can be tuned, along with the drug release rates. A skilled person in the art can determine the amount of vitamin K₂ and the size of the microsphere for different applications in various bone healing situations.

Also within the scope of this invention is a pharmaceutical composition that contains the vitamin K₂ microsphere described above and a pharmaceutically acceptable carrier including water, ethanol, and glycerol. The weight/volume ratio of the vitamin K₂ microsphere and the pharmaceutical acceptable carrier can be 0.005-75%.

The carrier in the pharmaceutical composition must be “acceptable” in the sense that it is compatible with the vitamin K₂ microsphere (and preferably, capable of stabilizing the microsphere) and not deleterious to the subject to be treated. One or more solubilizing agents can be utilized as pharmaceutical excipients for delivery of the vitamin K₂ microsphere.

Further disclosed is use of the vitamin K₂ microsphere thus prepared for the manufacture of a medicament to treat osteoporosis or repair damaged bone tissue.

Moreover, this invention covers a method of administering an effective amount of the vitamin K₂ microspheres described above to a patient in need thereof. “An effective amount” refers to the amount of the vitamin K₂ microspheres that is required to confer a therapeutic effect on the treated subject. Effective amounts, as recognized by those skilled in the art, depend upon the diseases to be treated, the route of administration, the excipient, and the possibility of co-usage with other therapeutic treatment.

To practice the method of the present invention, a composition having the above-described vitamin K₂ microspheres can be administered parenterally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Examples below illustrate surface morphology observations, particle size distributions, physical and chemical property analysis, and drug release tests of different concentrations of vitamin K₂ microspheres (VK₂MSs). Further, MG-63 cells are co-cultured with cell culture medium containing different concentrations of vitamin K₂ microspheres (VK₂MSs). The effects on the cell activity of vitamin K₂ microspheres (VK₂MSs) are observed by methods including cell viability assay (MTT assay), alkaline phosphatase activity assay (ALP activity assay) and immunohistochemical staining to find the most suitable vitamin K₂ microspheres (VK₂MSs) for applying to bone tissue repair engineering.

### PREPARATION EXAMPLES

### Preparation of Vitamin K₂ Microspheres (VK₂MSs)

Microspheres were prepared by the oil-in-water (O/W) emulsion nonaqueous phase separation method. 1.2 g of PVA was added to 60 ml of water and stirred under 100° C. for 30 minutes to obtain a 2% PVA solution. 0.001 g, 0.01 g, and 0.1 g of Vitamin K₂ (VK₂) and 0.2 g of PLGA were dissolved in 10 ml of dichloromethane and stirred in an ice bath at 1000 rpm for 10 minutes to form 0.01%, 0.1%, and 1% of VK₂ solutions.

PVA solution was poured into a 100 ml beaker in an ice bath. 10 ml of the VK₂ solution was slowly dropped into the PVA solution, stirred at 3200 rpm by a homogenizer, and then stirred at 5000 rpm for 10 minutes. Stirring was conducted in the hood at room temperature for 24 hours to remove dichloromethane. Then, larger microspheres were filtered by a cell sieve with 100 μm pore size. The obtained filtrate was poured into a 50 ml centrifuge tube and centrifuged at 2500 rpm for 10 minutes. After that, fresh deionized water was added.

The washing step using deionized water was repeated 4 times. Then, the VK₂ solution was poured into a microcentrifuge tube, rapidly cooled down by liquid nitrogen (N₂(I)), and dried by a freeze dryer for 24 hours. The obtained microspheres encapsulating VK₂ were abbreviated as VK₂MS. The product was weighed by an electronic microbalance to calculate the yield, stored in a drying cabinet and prepared for use.

### Example 1

### Analysis of the Property of the Microspheres

After the microspheres prepared by the oil in water (O/W) emulsion nonaqueous phase separation method were weighed by an electronic microbalance, an optical microscope (OM) and scanning electron microscope (SEM) were used to observe the surface morphology of the microspheres. Attenuated total reflectance-fourier transform infrared spectrometry (ATR-FTIR) was used to determine whether VK₂ was embedded in PLGA, A laser scattering particle size distribution analyzer (LS) was used to measure the particle size distribution. An ultraviolet-visible spectrophotometer (UV/Vis) was used to analyze the different encapsulation effects of the microspheres with different dosages embedded.

Surface Morphology Observation the of the Microspheres

After dichloromethane was removed, 0.5 ml of a solution containing microspheres was added into a microcentrifuge tube, and then 20 μl of that was dropped onto a hemocytometer and observed with an optical microscope (OM). The VK₂MS containing different concentrations of VK₂ were spherical, and uniformly dispersed without aggregation. Therefore, microspheres of uniform size were successfully formed by the oil in water (O/W) emulsion solvent distilling method.

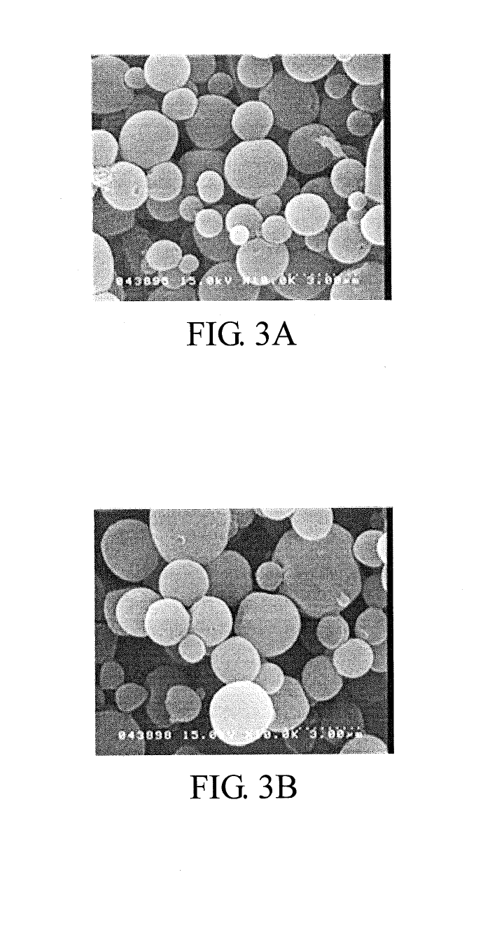

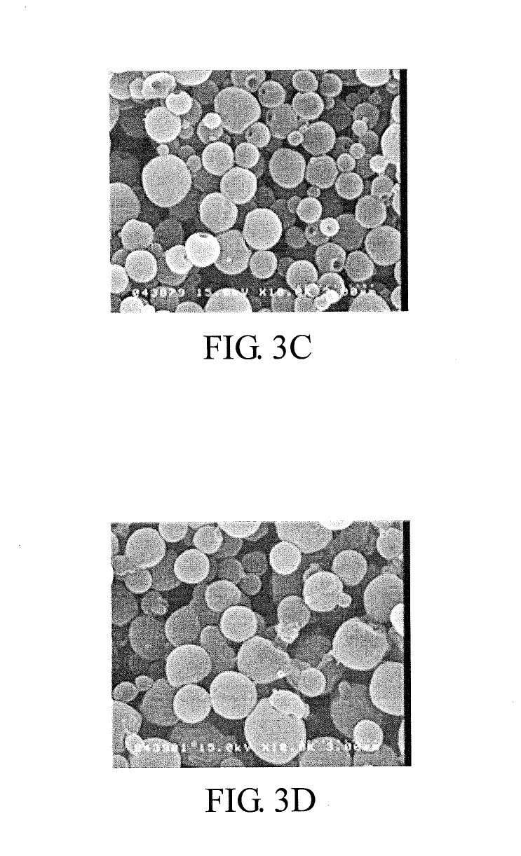

The freeze-dried microspheres were gently placed on a conductive tape, and those that did not adhere to the conductive tape were removed by a blowing ball. Then, after the surface of microspheres was platinized by an ion sputter by 15 mA for 3 minutes, the surface morphology was observed with a scanning electron microscope (SEM), as shown in FIGS. 3A-3D. In FIGS. 3A-3D, the surface of VK₂MS containing different concentrations of VK₂ was very smooth and appeared to be spherical without aggregation, which corresponds to the result of the optical microscope (OM).

Qualitative Analysis of the Microspheres

To determine whether the VK₂ was successfully embedded in PLGA and whether the surfactant of PVA was removed clearly, attenuated total reflectance-fourier transform infrared spectroscopy (ATR-FTIR) was used to confirm the existence of functional groups of VK₂ and PVA in the VK₂MS. The freeze-dried microspheres were observed by ATR-FTIR to observe the absorption spectrum of VK₂MS. (Number of scans: 128, Resolution: 8, Wavenumber: 4000-650 cm⁻¹)

Generally, PLGA has —OH stretching vibration at 3200-3500 cm⁻¹, —CH stretching at 2850-3000 cm⁻¹, —C═O stretching vibration at 1700-1800 cm^{—1}, and C—O stretching at 1050-1250 cm⁻¹. PVA has a broader O—H stretching vibration at 3100-3400 cm⁻¹, and C—H stretching vibration at 2930 cm⁻¹. VK₂ has C═C stretching vibration at 1500-1600 cm⁻¹, C═O stretching at 1690-1760 cm⁻¹, and C—H stretching vibration at 3010-3100 cm⁻¹.

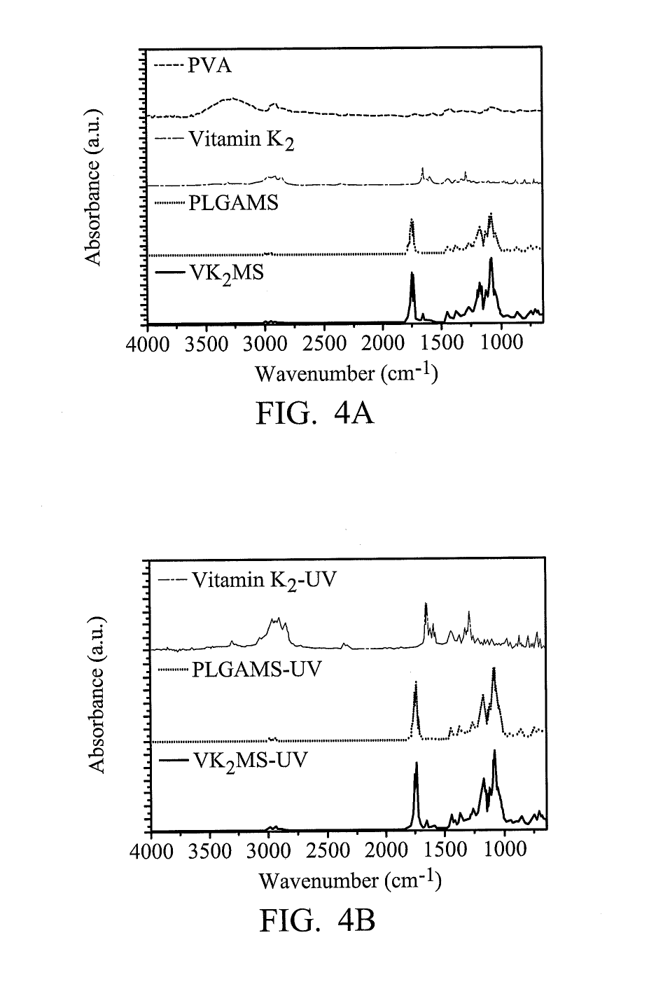

As shown in FIG. 4A, the peak of the O—H functional group at 3100-3400 cm⁻¹ was significantly lower, which illustrated that PVA remaining on the microspheres was substantially removed. VK₂MS was observed to have C═C stretching vibration at 1500-1600 cm⁻¹, which indicates that there was a VK₂ functional group. Accordingly, VK₂ was proved to be encapsulated in the microspheres.

Since cell experiments have to be operated under sterility, the materials were co-cultured with cells after sterilization by UV light. However, to prevent chemical reactions from occurring after the UV irradiation, the materials irradiated by UV light were analyzed by ATR-FTIR to determine whether the positions of the functional groups had changed, as shown in FIG. 4B.

According to FIG. 4B, the positions of functional groups of materials irradiated by UV light had not changed, and were the same as the characteristic absorption peaks shown in FIG. 4A, which proved that the properties of the materials used in the present experiments would not change after being irradiated by UV light, and therefore, VK₂MS could be used in the subsequent cell experiments after being sterilized by UV light.

Particle Size Analysis of the Microspheres

5 mg of microspheres were added into 5 ml of deionized water and oscillated in the ultrasonic oscillator to uniformly disperse the microspheres. Then, a laser scattering particle size distribution analyzer (LS) was used to analyze the particle size distribution of the microspheres and calculate the span.

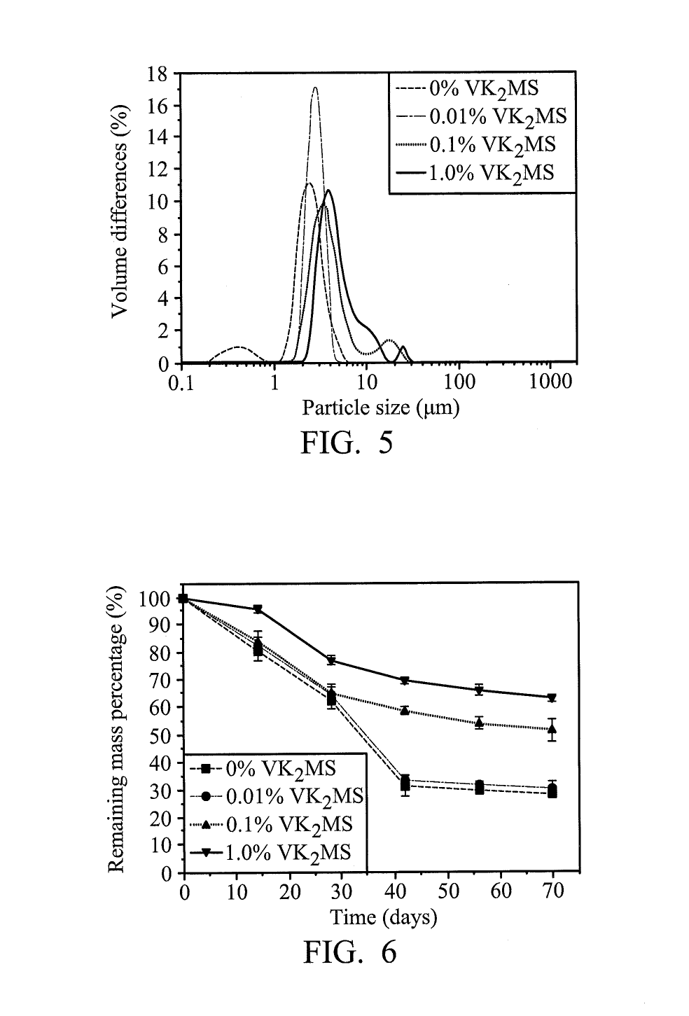

As illustrated in FIG. 5, it was observed that the particle size distribution of 0.01% VK₂MS is more narrow, which represents a more uniform size of microspheres. The result corresponds to the smaller span of 0.5 of 0.01% VK₂MS shown in Table 1, while 0.1% VK₂MS has a larger span of 1.6. The larger span represents a wider particle size distribution range, and a less uniform size of microspheres. The smaller span represents a more narrow particle size distribution range, and a more uniform size of microspheres.

TABLE 1

| | | | | 1% |
|---|---|---|---|---|
| | 0% VK₂MS | 0.01% VK₂MS | 0.1% VK₂MS | VK₂MS |
| Particle size | 2.5 ± 1.0 | 2.9 ± 0.6 | 5.2 ± 4.4 | 5.9 ± 4.0 |
| (μm) | | | | |
| Span | 1.0 | 0.5 | 1.6 | 1.5 |

According to Table 1, it seems that the particle size is related to the content of VK₂. The average particle size increased with the increasing encapsulated amount of vitamin K₂ in the microspheres. The largest particle size was 5.9±4.0 μm of 1.0%

VK₂MS, which was consistent to the observation of SEM. In this particle size range, the microspheres neither fall out of the bracket when placed in tissue engineering scaffolds since the particle size is too small nor affect the uniform drug release to result in difficulty controlling the drug release due to the particle size being too large.

### Example 2

### In Vitro Drug Release

Preparation of Phosphate Buffer Saline (PBS)

8 g of NaCl, 0.2 g of KCl, 2.16 g of Na₂HPO₄, 0.2 g of KH₂PO₄, and 1000 ml of water were added into a bottle and stirred until completely dissolved. Then, the pH value of a solution was adjusted to 7.4. The bottle was autoclaved at 115° C. for 30 minutes and then cooled down under room temperature.

Degradation Experiments of the Microspheres

0.01 g of freeze-dried 0%, 0.01%, 0.1%, and 1.0% VK₂MS were added into 15 ml centrifuge tubes, 3 ml PBS was added, then the centrifuge tubes were placed in a 37° C. water bath for drug release for 0, 14, 28, 42, 56, and 70 days. 30 minutes before the sampling time, the centrifuge tube rack was taken from the water bath and left to stand for 30 minutes to precipitate the microspheres. PBS was removed from the centrifuge tubes and the microspheres were placed into microcentrifuge tubes and solidified in a −20° C. refrigerator for 2 hours. After being dried by a freeze dryer for 24 hours, the product was weighed by an electronic microbalance.

In the present disclosure, VK₂ drug was dispersed into a biodegradable polymer matrix, and could diffuse from the matrix or be released by polymer dissolution. Accordingly, the degradation rate of polymer had a great impact on the drug release rate.

In FIG. 6, the hydrolysis rate of 0.01% VK₂MS was almost as fast as that of 0% VK₂MS, after 42 days, the remaining mass percentages were respectively 33.0±2.2% and 31.0±3.7%. The remaining mass percentage of 0.1% VK₂MS and 1.0% VK₂MS were 58.7±1.2% and 69.3±1.2%, respectively. The degradation rate became slower with the increasing encapsulated amount of VK₂. In particular, the degradation rate of the 1.0% VK₂MS was the slowest, the remaining mass percentage of which was 62.7±1.2 after the 70-day degradation experiment. The reason for this phenomenon can be hydrophobic drugs VK₂, which can hinder the hydrolysis of PLGA. The more drugs encapsulated, the greater the obstruction and slower the degradation rate became.

VK₂ Drug Release

0.01 g of freeze-dried 0%, 0.01%, 0.1%, and 1.0% VK₂MS were added into 15 ml centrifuge tubes, 3 ml PBS was added, then the centrifuge tubes were placed in a 37° C. water bath for drug release for 0, 1, 3, 7, 14, 21, 28, 35, 42, 49, 56, 63, and 70 days. 30 minutes before the sampling time, the centrifuge tube rack was taken from the water bath and standing for 30 minutes to precipitate the microspheres. Then, 2.5 ml of the supernatant was suctioned from the centrifuge tube to a 20 ml glass vial and 2.5 ml of fresh PBS was supplied into the centrifuge tube to maintain the solution volume at 3 ml. Then, the centrifuge tubes were placed back to the 37° C. water bath.

20 ml of glass vial containing 2.5 ml of different supernatants were placed in a −20° C. refrigerator to solidify for 2 hours. After dried by a freeze dryer for 24 hours, 5 ml of dichloromethane was added into a 20 ml glass vial and stirred at 600 rpm for 10 minutes. Then, the sample was suctioned by a glass syringe to a 0.45 μm-syringe filter. The obtained filtrate was observed by an ultraviolet-visible spectrophotometer (UVIVis) at 320 nm to analyze the content of VK₂.

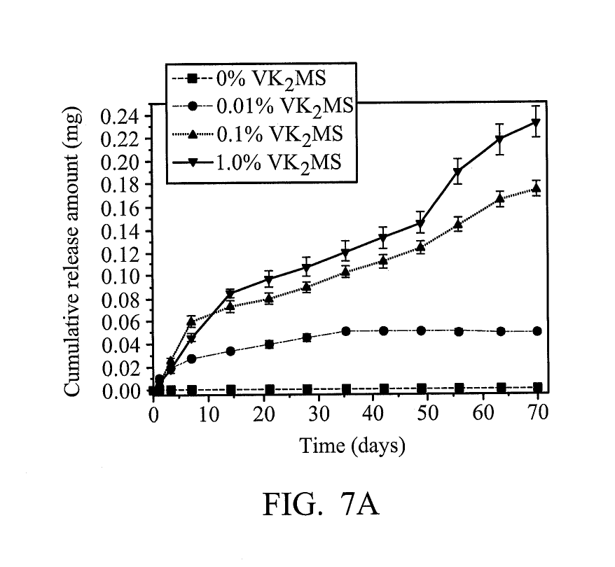

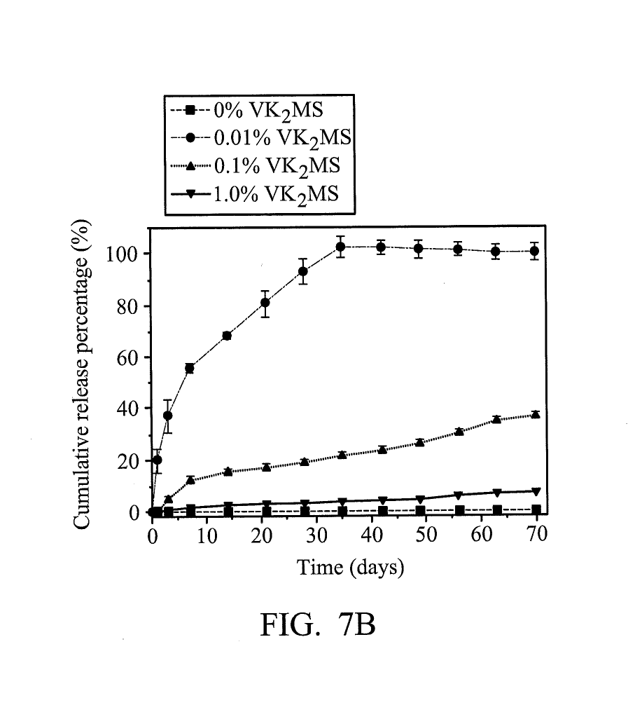

The cumulative release amount of VK₂MS encapsulated with different concentrations of VK₂ is illustrated in FIG. 7A. On day 1, 0.01% VK₂MS released more VK₂ (0.0098±0.0024 mg). However, on day 3, 0.1% VK₂MS cumulatively released more VK₂ (0.0240±0.0042 m). After 14 days, the cumulative release amount of 1.0% VK₂MS (0.0846±0.0033 mg of VK₂), which originally released only slowly, surpassed the cumulative release amount of 0.1% VK₂MS (0.0733±0.0051 mg VK₂). Similarly, after a 35 day-cumulative release amount of 0.0510±0.0021 mg of VK₂, the curve of the cumulative release amount of 0.01% VK₂MS became flat with no rising trend.

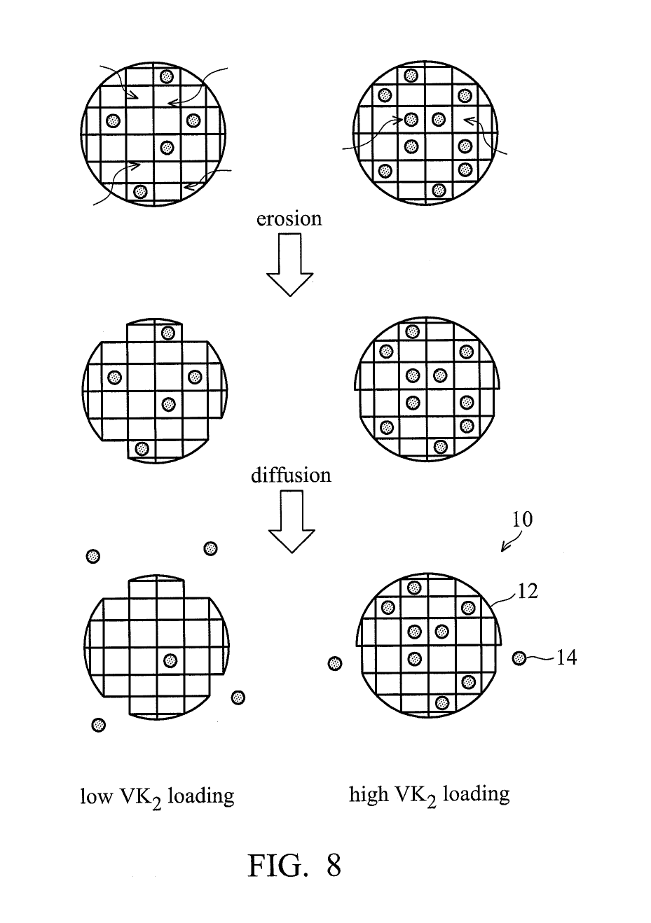

The cumulative release percentage can be obtained by dividing the cumulative drug release amount by the theoretical drug-coated amount, as shown in FIG. 7B. According to FIG. 7B, after 35 days, 0.01% VK₂MS reached 100% release, while the 0.1% VK₂MS and 1.0% VK₂MS still slowly released drug and respectively released 36.6±1.4% and 7.0±0.4% of VK₂ on day 70. It revealed that the drug release rate became slower with the increasing drug loading. In particular, the release rate of 1.0% VK₂MS was slower, which was not only related to the erosion rate of the polymer surface but also closely related to the content of VK₂ as well as its diffusion rate. With a higher content of VK₂, the hydrolysis of PLGA was hindered and a longer release time was needed. With a lower VK₂ content, there was no hindrance and the hydrolysis of PLGA was unaffected, such that the drug release time became short. Since the result corresponds to FIG. 8, the drug release rate proved to be related to the degradation rate.

FIG. 8 is a schematic diagram of the release of VK₂ 14 by VK₂MS 10. According to the drug release curve of FIG. 7B, 0.01% VK₂MS complies with the zero order drug release kinetics mode. That is, the drug release rate had nothing to do with the drug concentration, but was only related to the reaction rate constant k. The present disclosure mainly investigates the VK₂ release condition of VK₂MS containing different encapsulated concentrations. Usually, there are three conditions of drug release routes: at the beginning, the drug releases near the surface of the microspheres; at the middle stage, the surface of the microspheres is eroded and pores are produced, and drugs are released from the inter-pores; at the last stage, the drug diffuses through a matrix. In accordance with the experimental results, the encapsulated amount of VK₂ 14 affects the degradation rate and indirectly affects the drug release rate, and therefore is one of the important parameters affecting the controlled drug release rate.

### Example 3

### Human Osteosarcoma Cell Line (MG-63) Activity Analysis

Preparation of High Glucose-Dulbecco's Modified Eagle Medium (H-DMEM) Culture Medium

Bottles containing deionized water, beakers, and stir bars were autoclaved at 115° C. for 30 minutes for sterilization. 3.75 g of sodium bicarbonate was fine weighed and prepared for use. H-DMWM powder was poured in a 1000 ml beaker containing 900 ml deionized water in a sterile hood. After the solution was uniformly stirred by a stir bar, 3.75 g of sodium bicarbonate was added and stirred until completely dissolved.

A pH meter was used to measure the pH value of the solution, and the pH value was adjusted to 7.26 by accessing CO₂. H-DMEM solution was filtered by 0.22 μm sterile filtration equipment and poured into the sterilized bottle. Then, 100 ml of fetal bovine serum (FBS), which was deactivated at 56° C. for 30 minutes and 10 ml of PSA, were added. The sealed bottle was kept in a 4° C. refrigerator. The product was H-DMEM culture medium containing 10% FBS.

MG-63 Cells Culture and Sub-Culture

A human osteosarcoma cell line (osteoblast-like cell line) MG-63, purchased from Food Industry Research and Development Institute (FRIDI) was used in the present disclosure.

H-DMEM containing 10% FBS was used to adjust the concentration of MG-63 cell solution to 1×10⁵ cells/ml. 5 ml of cell solution was added into a T-25 flask and incubated in an incubator at 37° C., 5% CO₂, and 95% R.H. H-DMEM containing 10% FBS was changed every two days. An inverted microscope (Olympus, CKX31) was used to observe the growth of MG-63 cells. When MG-63 cells were about 80%-full in the flask, MG-63 cells could be sub-cultured. The cell passage of MG-63 cells was 8-24.

Preparation of Cell Culture Medium with Different VK₂ Concentration

0.0001 g, 0.0005 g, 0.005 g, and 0.05 g of VK₂ was fine weighed and sterilized by UV light in a sterile hood overnight, then added into 50 ml centrifuge tubes. 50 ml of H-DMEM containing 10% FBS was then added to prepare cell culture medium with different VK2 concentrations: 0 mg/mL, 0.002 mg/mL, 0.01 mg/mL, 0.1 mg/mL, and 1 mg/mL.

Co-Culture Cells and Cell Culture Medium Containing Different VK₂ Concentration

H-DMEM containing 10% FBS was used to adjust the concentration of MG-63 cell solution to 1×10⁴ cells/ml. 1 ml/well of cell solution was seeded to a 24-well plate and incubated in an incubator for 1 day to make cells adhere. Then, the culture medium was removed, 1 ml/well of cell culture medium with different VK₂ concentration were added respectively and incubated for 1, 3, and 7 days. Cell viability analysis (MTT) and alkaline phosphatase activity assay (ALP) were conducted. Cell morphology was observed by an inverted microscope and recorded in photographs.

Preparation of Cell Culture Medium with Different VK₂MS

0.01 g of 0%, 0.01%, 0.1%, and 1.0% VK₂MS was fine weighed and sterilized by UV light in a sterile hood overnight, then added into 50 ml centrifuge tubes. 50 ml of H-DMEM containing 10% FBS was then added to prepare cell culture medium with different VK₂ concentrations.

Implant Cell Culture Medium Containing VK₂MS with Different VK₂ Concentration Into Cells

H-DMEM containing 10% FBS was used to adjust the concentration of MG-63 cell solution to 1×10⁴ cells/ml. 1 ml/well of cell solution was seeded to a 24-well plate and incubated in an incubator for 1 day to make the cells adhere. Then, the culture medium was removed, 1 ml/well of cell culture medium with different VK₂ concentration were added respectively and incubated for 1, 3, and 7 days. In addition, the cell culture medium without microspheres (MS) was added 1 ml/well and incubated for 1, 3, and 7 days as control groups. Subsequently, cell viability analyse (MTT) and alkaline phosphatase activity assays (ALP) were conducted. Cell morphology was observed by an inverted microscope and recorded by photographs.

Moreover, H-DMEM was used to adjust the concentration of MG-63 cell solution to 1×10⁴ cells/ml. 1 ml/well of cell solution was seeded to a 24-well plate and incubated in an incubator for 1 day to make cells adhere. Then, the culture medium was removed, 1 ml/well of cell culture medium containing microspheres with different VK₂ concentration were added respectively and incubated for 1, 3, and 7 days. Then, histochemical staining analysis was conducted by H&E, Von Kossa, and Alizarin red.

Cell Viability Analysis

MTT (3-(4,5-Dimethylthiazol-2-yl)-2,5-diphenyltetrazolium bromide) is a tetrazolium salt which reveals mitochondrial dehydrogenase of live cells and capable of reducing mitochondrial dehydrogenase of live cells to blue formazan crystals. When the cell number increases or the cell proliferation is good, the vigorous function of cellular mitochondria results in increasing mitochondrial dehydrogenase. Accordingly, blue formazan crystals formed after a reaction with MTT also increases, which can be a quantitative basis of the cellular mitochondria activity.

First, MTT was prepared as a 5 mg/mL reaction solution by PBS, then filtered by a 0.22 μm filter. 10% MTT reagent was prepared by H-DMEM, and then stored in the dark. After being cultured for 1 day, the cell culture medium was removed from the 24-well cell culture plate. The 24-well cell culture plate was washed twice by PBS. After the PBS was removed, 1 ml/well of 10% MTT reagent was added to the culture plate and reacted in an incubator (37° C., 5% CO₂) in the dark for 4-5 hours. Purple crystal particles were produced at the bottom of culture plate after the reaction. The reactive solution was removed and 350 μl/well of DMSO solution was added. After being mixed, 1000/well of dissolved solution was suctioned to a 96-well plate and the absorbance at 570 nm was detected by an ELISA reader. The reference wavelength was set as 650 nm.

A cell standard curve can be made to reckon the cell number. The cell solution was adjusted to have a concentration of 5000, 10000, 25000, 50000, 75000, and 100000 cells/ml by H-DMEM containing 10% FBS then seeded into a 24-well plate, respectively. After 1 day, after the cells were adhered, an MTT cell viability analysis was conducted. Absorbance and cell numbers were used to make a standard curve, such that the cell number could be reckoned from the absorbance.

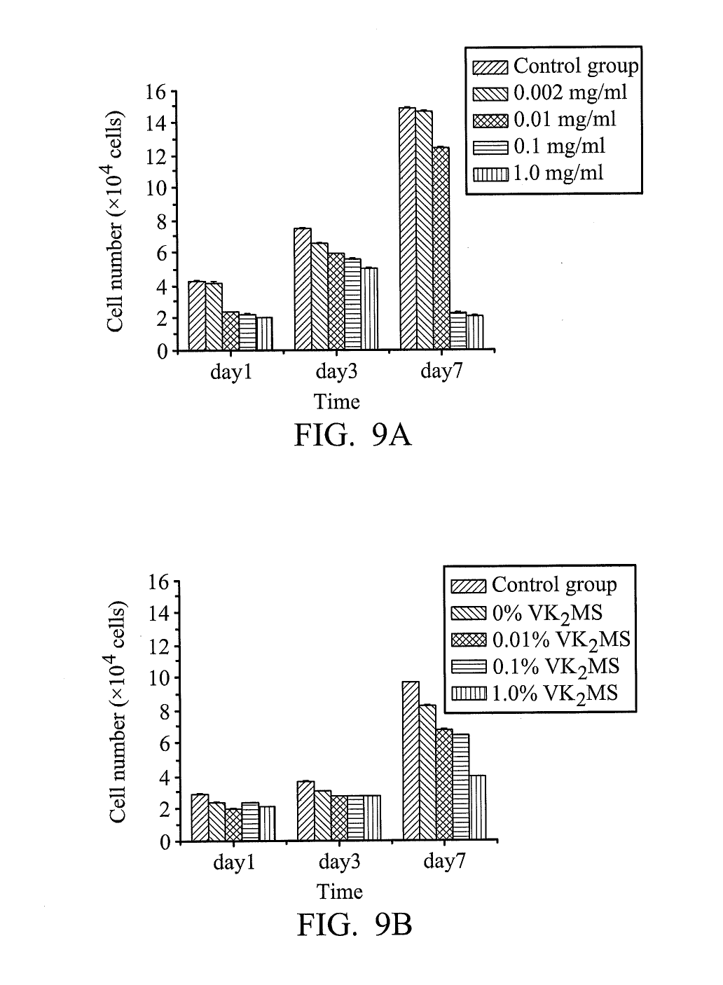

A growth curve of MG-63 cells was made first, and then the cell growth rate could be obtained by dividing the cell viability by that of the control group which was detected by the MTT assay. The result shown in FIG. 9A can be obtained by converting the data from ELISA to a cell number according to the cell growth curve. In FIG. 9A, it is observed that the cell number of MG-63 co-cultured with VK₂ is apparently lower than that of the control group. Moreover, the effect of cell growth inhibition becomes obvious with the increasing concentration of VK₂. During the co-culture experiment of VK₂ and MG-63 cells, the highest number of cells (4.1±0.1×10⁴ cells) appeared in the group of 0.002 mg/mL of VK₂ and the lowest cell number (2.0±0.0×10⁴ cells) appeared in the group of 1 mg/mL of VK₂ on day 1; the highest cell number (6.6±0.1×10⁴ cells) appeared in the group of 0.002 mg/mL of VK₂ and the lowest cell number (5.0±0.1×10⁴ cells) appeared in the group of 1 mg/mL of VK₂ on day 3; the highest cell number (14.7±0.1×10⁴ cells) appeared in the group of 0.002 mg/mL of VK₂ and the lowest cell number (2.1±0.1×10⁴ cells) appeared in the group of 1 mg/mL of VK₂ on day 7. The result corresponded to FIG. 31. It was proved that VK₂ inhibits cell proliferation, and that the cellular survival rate reduces with the increasing concentration of VK₂.

Similarly, the result in FIG. 9B can be obtained by converting the data from ELISA to cell number according to the cell growth curve. In FIG. 9B, it is observed that effect of cell growth inhibition is also achieved when MG-63 cells are co-cultured with different concentrations of VK₂MS; however, the inhibition effect is not obvious, which reveals that VK₂MS possess the effect of delaying release. The cell number of MG-63 co-cultured with VK₂ MS is apparently lower than that of control group. However, the cell number increased with the increasing number of days, representing a less obvious inhibition effect. During the co-culture experiment of VK₂MS and MG-63 cells, the highest cell number (2.4±0.0×10⁴ cells) appeared in the group of 0 mg/mL of VK₂MS and the lowest cell number (2.1±0.0×10⁴ cells) appeared in the group of 0.01 mg/mL of VK₂MS on day 1; the highest cell number (3.1±0.0×10⁴ cells) appeared in the group of 0 mg/mL of VK₂MS and the lowest cell number (2.8±0.0×10⁴ cells) appeared in the group of 1.0 mg/mL of VK₂MS on day 3; the highest cell number (8.4±0.1×10⁴ cells) appeared in the group of 0 mg/mL of VK₂MS and the lowest cell number (4.0±0.0×10⁴ cells) appeared in the group of 1.0 mg/mL of VK₂MS on day 7.

Alkaline Phosphatase Activity Test

Alkaline phosphatase (ALP) is a glycoprotein compiled by many gene groups. Many scholars believe that ALP facilitates hydrolysis of phosphomonoesters and releases phosphate ions, which in turn induce the mineralization of matrix outside osteoblasts, i.e., bone-forming cells. As such, the activity of alkaline phosphatase (ALP) is used as a biological indicator for the activity of osteoblasts and the basis of bone cell differentiation.

The measurement of ALP is described below. After being cultured for 1 day, the cell culture medium was removed from the 24-well cell culture plate. Then, the 24-well cell culture plate was washed twice by PBS. After the PBS was removed, 200 μl/well of ALP extraction reagent pNPP65 (p-Nitrophenylphosphate) was added to the 24-well culture plate and reacted in an incubator (37° C., 5% CO₂) in the dark for 30 minutes. Then, 50 μl of 1N sodium hydroxide was added to stop the reaction. 250 μl/well of supernatant was suctioned to a 96-well plate to detect the OD value at 405 nm by an ELISA reader. Activity of alkaline phosphatase (ALP) can be calculated by the formula below:

$$ALP ( U / I ) = \frac{A \times Vt \times 1000}{t \times ɛ \times l \times Vs}$$

In this formula, A is the absorbance of a sample at 405 nm; Vt is the total reaction volume, 0.25 ml; Vs is the sample volume, 0.05 ml; t is the reaction time after adding pNPP65 (p-Nitrophenylphosphate), 30 minutes; c is mmol extinction coefficient of pNPP65 (p-Nitrophenylphosphate), 18.6 mM⁻¹ cm⁻¹; 1 is the optical path of the cuvette, 1 cm; 1000 is to convert the U/ml to U/l.

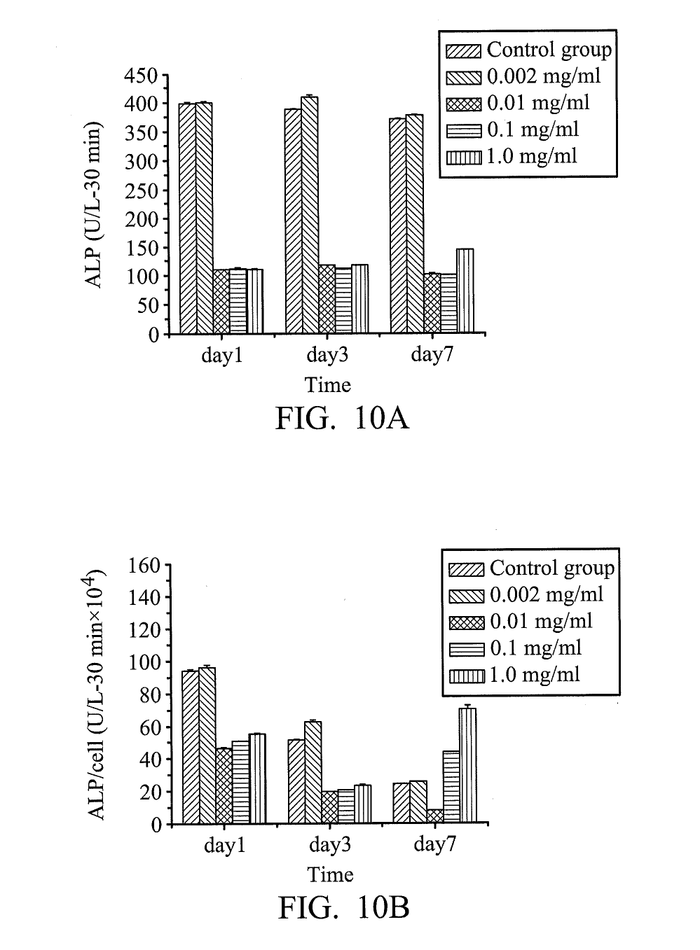

Alkaline phosphatase (ALP) is regarded as biological indicators of the activity of osteoblasts and the basis of bone cell differentiation. According to FIG. 10A, it was observed that the ALP value of MG-63 co-cultured with 0.002 mg/mL of VK₂ is higher than that of the control group, while the ALP values of other groups are lower than that of control group. During the co-culture experiment of VK₂ and MG-63 cells, the highest ALP value (399.9±3.3 U/L-30 min) appeared in the group of 0.002 mg/mL of VK₂ and the lowest ALP value (111.0±0.7 U/L-30 min) appeared in the group of 0.01 mg/mL of VK₂ on day 1; the highest ALP value (410.7±2.7 U/L-30 min) appeared in the group of 0.002 mg/mL of VK₂ and the lowest ALP value (112.2±0.3 U/L-30 min) appeared in the group of 0.1 mg/mL of VK₂ on day 3; the highest ALP value (377.4±1.6 U/L-30 min) appeared in the group of 0.002 mg/mL of VK₂ and the lowest ALP value (100.6±0.0 U/L-30 min) appeared in the group of 0.1 mg/mL of VK₂ on day 7. It can be deduced that a trace amount of VK₂ was needed for facilitating the differentiation of osteoblasts. Conversely, the effect of large amount of VK₂ was poor and ALP activity of cells was reduced.

Alkaline phosphatase activity of a single cell can be obtained by dividing the detected ALP activity by the cell number, as shown in FIG. 10B. In FIG. 10B, it was observed that the value of 0.002 mg/mL of VK₂ was higher than that of the control group on day 1; however, all of the values decreased on day 3 since the cells were still increasing while the ALP did not increase so much. However, on day 7, due to the increased ALP activity of 1 mg/mL of VK₂ and the decreased cell number, the values were raised. During the co-culture experiment of VK₂ and MG-63 cells, the highest ALP activity of a single cell (96.7±1.1 U/L-30 min-10⁴ cells) appeared in the group of 0.002 mg/mL of VK₂ and the lowest ALP activity of a single cell (46.4±0.4 U/L-30 min-10⁴ cells) appeared in the group of 0.01 mg/mL of VK₂ on day 1; the highest ALP activity of a single cell (62.4±0.9 U/L-30 min-10⁴ cells) appeared in the group of 0.002 mg/mL of VK₂ and the lowest ALP activity of a single cell (19.8±0.1 U/L-30 min-10⁴ cells) appeared in the group of 0.01 mg/mL of VK₂ on day 3; the highest ALP activity of a single cell (70.4±1.7 U/L-30 min-10⁴ cells) appeared in the group of 1.0 mg/mL of VK₂ and the lowest ALP activity of single cell (8.2±0.0 U/L-30 min-10⁴ cells) appeared in the group of 0.01 mg/mL of VK₂ on day 7.

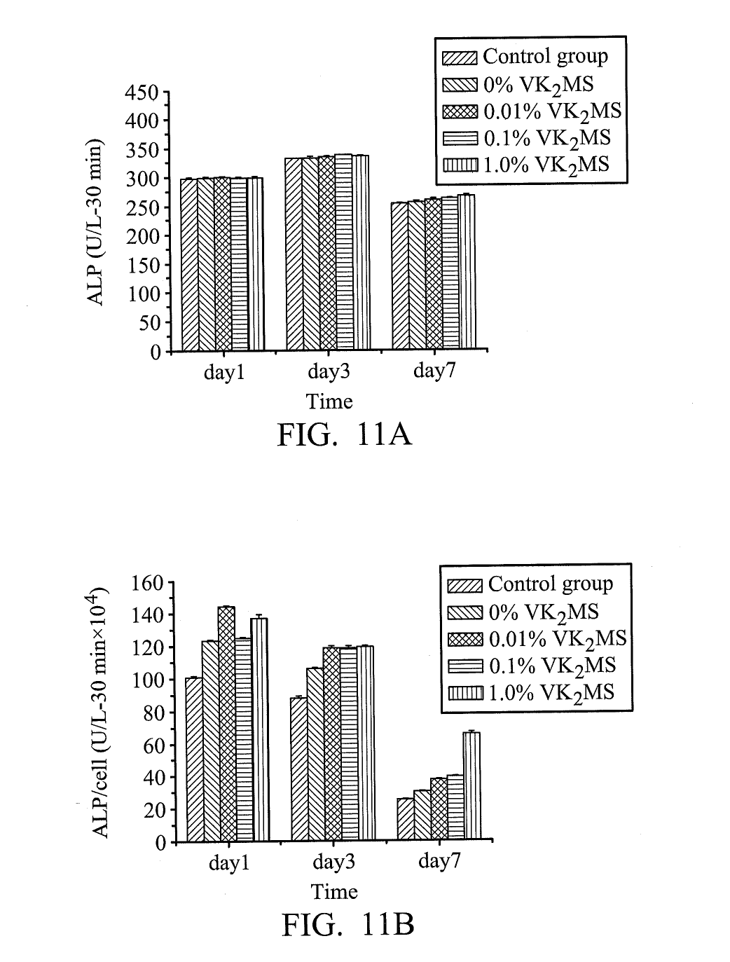

In FIG. 11A, it was observed that VK₂MS is capable of enhancing the ALP activity of MG-63 cells. However, the AKP value decreases on day 7. The reason can be that cells cannot proliferate and differentiate at the same time; therefore, the ALP value decreased because cells were proliferating on day 7. During the co-culture experiment of VK₂MS and MG-63 cells, the highest ALP activity (299.5±1.8 U/L-30 min) appeared in the group of 0.01% of VK₂MS and the lowest ALP activity (296.9±3.0 U/L-30 min) appeared in the group of 1.0% of VK₂MS on day 1; the highest ALP activity (336.7±1.0 U/L-30 min) appeared in the group of 0.1% of VK₂MS and the lowest ALP activity (331.6±2.9 U/L-30 min) appeared in the group of 0% of VK₂MS on day 3; the highest ALP activity (265.8±3.2 U/L-30 min) appeared in the group of 1.0% of VK₂MS and the lowest ALP activity (254.8±2.8 U/L-30 min) appeared in the group of 0% of VK₂MS on day 7.

Similarly, alkaline phosphatase activity of a single cell can be obtained by dividing the ALP activity by the cell number, as shown in FIG. 11B. In FIG. 11B, it was observed that the value of MG-63 cells co-cultured with VK₂MS was higher compared to that of the control group. In particular, the highest value appeared in the group of 0.01% of VK₂MS on day 1. Such condition is related to the higher release amount of VK₂ of 0.01% VK₂MS in the initial in vitro release stage, so VK₂MS can effectively increase the ALP activity of MG-63 cells compared to the control group, and make single cell possess higher ALP value. During the co-culture experiment of VK₂MS and MG-63 cells, the highest ALP activity of a single cell (144.9±0.4 U/L-30 min-10⁴ cells) appeared in the group of 0.01% of VK₂MS and the lowest ALP activity of a single cell (123.4±1.0 U/L-30 min-10⁴ cells) appeared in the group of 0% of VK₂MS on day 1; the highest ALP activity of a single cell (119.6±0.3 U/L-30 min-10⁴ cells) appeared in the group of 1.0% of VK₂MS and the lowest ALP activity of a single cell (106.5±0.4 U/L-30 min-10⁴ cells) appeared in the group of 0% of VK₂MS on day 3; the highest ALP activity of a single cell (66.5±1.0 U/L-30 min-10⁴ cells) appeared in the group of 1.0% of VK₂MS and the lowest ALP activity of a single cell (30.5±0.6 U/L-30 min-10⁴ cells) appeared in the group of 0% of VK₂MS on day 7.

To sum up the above, the concentration of VK₂ released from VK₂MS will affect the MG-63 cell growth rate and the differentiation of the activity of ALP. The higher concentration of VK₂ released, the more obvious that cell growth rate is inhibited; however, the activity of differentiating ALP of cells is enhanced. Consequently, the growth characteristics of cells are affected by the changes of VK₂ concentration released to the culture medium by degradation of VK₂MS.

### Example 4

### Histochemical Staining of MG-63 Cells

Alizarin Red S Stain

Alizarin red S, which appears bright red when combined with calcium, is a red dye usually used in histochemical staining to determine whether calcium deposits. Also, it is usually used as a basis for determining the mineral deposition of mineralization nodules formed by accumulation of osteoblasts.

First, 50 mL of deionized water was added into a 50 mL centrifuge tube containing 2 g of paraformaldehyde to prepare a 4% (w/v) paraformaldehyde solution. 50 mL of deionized water was added into a 50 ml centrifuge tube containing 1 g of alizarin red S to prepare a 2% (w/v) alizarin red S solution.

A cell culture medium in wells was suctioned. The wells were washed three times by PBS. After the PBS was suctioned, a 4% paraformaldehyde solution was added and reacted for 30 minutes to fix cells and then was suctioned. The wells were washed three times for 5 minutes each time with deionized water and then suctioned. 2% alizarin red S solution was added for 10 minutes and then suctioned. Washed three times by deionized water, 5 minutes for each time, then deionized water was suctioned. The results of the staining were observed under an inverted microscope and saved in photographs.

It can be found from the experimental results that when MG-63 cells were co-cultured with VK₂MS, more bright red calcium was deposited. Moreover, the calcium deposition increased not only with the increasing loading of VK₂, but also with increasing days.

Hematoxylin & Eosin Stain

Hematoxylin & Eosin stain is a routine staining, which uses two colouring agents of hematoxylin and eosin to distinguish cytoplasm and cell nucleus. Hematoxylin is a basic dye that specifically used to stain cell nucleus and basophilic cells, and appears violet after combined with nucleic acid in the cell nucleus. Eosin is an acid dye that specifically used to stain cytoplasm and acidophil cells, and appears pink after combined with protein in the cytoplasm.

First, 50 ml of deionized water was added into a 50 ml centrifuge tube containing 0.25 g of eosin to prepare a 0.5% (w/v) eosin solution. Every time before used, the pH value of solution has to be adjusted to 4.1-4.3 by glacial acetic acid.

A cell culture medium in wells was suctioned. The wells were washed three times by PBS. After the PBS was suctioned, a 4% paraformaldehyde solution was added and reacted for 30 minutes and then was suctioned. The wells were washed three times by deionized water for 5 minutes each time and then deionized water was suctioned. Harris alum hematoxylin was added for 5 minutes and then suctioned. Sample was washed by deionized water until bright violet appeared, then deionized water was suctioned. Then, sample was soaked in 0.5% eosin solution for 5 minutes (dipped in alcohol when the color was too dark). Washed three times by deionized water for 5 minutes each time and then deionized water was suctioned. The results of the staining were observed under an inverted microscope and saved in photographs.

It can be found from the experimental results that the cell number of control group is higher with more violet cell nucleus and pink cytoplasm. Moreover, the number of color increased with time. With respect to the co-culture of MG-63 cells and VK₂MS, the cell number decreased with the increasing concentration of VK₂. In addition, the cell number of 1.0% VK₂MS group was apparently fewer on day 7.

Von Kossa Stain

Since most of the calcium ions within tissues present in the form of calcium phosphate or calcium carbonate, this method replaces calcium ions with silver ions from a silver nitrate solution to form silver phosphate or silver carbonate. By the reduction reaction of silver ions, silver phosphate or silver carbonate appears to have a brownish-black color, such that the existence of calcium phosphate or calcium carbonate can be proven. Cells appear to have a pink color.

50 ml of deionized water was added into a 50 ml centrifuge tube containing 2.5 g of AgNO₃ to prepare a 5% (w/v) silver nitrate solution. 50 ml of deionized water was added into a 50 ml centrifuge tube containing 2.5 g of sodium thiosulfate to prepare a 5% (w/v) sodium thiosulfate solution.

A cell culture medium in wells was suctioned. The wells were washed three times by PBS. After the PBS was suctioned, a 4% paraformaldehyde solution was added and reacted for 30 minutes and then suctioned. The wells were washed three times by deionized water for 5 minutes each time and then deionized water was suctioned. After 5% sodium thiosulfate solution was added, sample was irradiated by UV light for 1 hour to convert Ca²⁺ to Ag⁺. Washed three times by deionized water. 5% sodium thiosulfate solution was added for 5 minutes to remove the remaining silver nitrate. After being washed three times by deionized water, nuclear fast red was added for 5 minutes as a comparison. Washed three times by deionized water for 5 minutes each time and then deionized water was suctioned. The results of the staining were observed under an inverted microscope and saved in photographs.

It can be found from the experimental results that when MG-63 cells were co-cultured with VK₂MS, more brownish-black calcium was deposited. Moreover, the number of color increased with time, which was consistent with the results shown in FIG. 14. The number of pink cells decreased with the increasing encapsulated loading of VK₂. In addition, the cell number of 1.0% VK₂MS group was apparently fewer on day 7.

The results shown in histochemical staining are the same as that of alkaline phosphatase activity assay. For example, using the alizarin red S stain, more bright red calcium ions were deposited in cells when VK₂MS was added; using an H&E stain, when VK₂MS was added, cell proliferation was inhibited and the number of violet cell nuclei and pink cytoplasms decreased; using a Von Kossa stain, more brownish black calcium was deposited in cells when VK₂MS was added, while the number of pink-cell nuclei apparently decreased.

The present disclosure successfully prepares VK₂MS with 0%, 0.01%, 0.1%, and 1.0% of VK₂ encapsulated by biodegradable polymer PLGA which is formed by the oil in water (O/W) emulsion nonaqueous phase separation method. The VK₂MS has a sleek spherical appearance without aggregation. The highest production of 1.0 VK₂MS reaches 80.8±6.9%, and the best encapsulation efficacy reaches 92.8±5.2%. The microspheres have an uniform particle-size distribution and an average particle size between 1 μm and 150 μm.

By the in vitro drug release experiment, it was found that the release curve of 0.01% VK₂MS complies with the zero order kinetics mode, which is beneficial in delaying release and stably controlling the drug concentration released to the outside.

0.01% VK₂MS completely releases VK₂ after 35 days, which is consistent with the faster degradation rate of 0.01% VK₂MS found in the degradation experiment. Moreover, it was found that a higher encapsulated amount of VK₂ will hinder the hydrolysis of PLGA and decrease its degradation rate, causing a lower drug release rate.

In the cell culture tests, it was found that the growth of MG-63 cells is inhibited by VK₂, and an inhibition effect becomes obvious with the increasing concentration of VK₂. However, 0.02 mg/mL can enhance the ALP activity of cells and result in a higher ALP activity in a single cell. Proliferation of MG-63 cells is also inhibited by VK₂MS; however, the effect is less than that of VK₂, representing a delayed release effect of VK₂MS. VK₂MS especially can efficiently enhance the ALP activity of a single cell.

Since the in vitro drug release rate of VK₂MS is affected by the degradation rate, the in vitro cell experiment was also interfered with by the same factor. It takes a short time, one week, for the in vitro cell tests in the present invention, while it takes 3-4 months to repair bone tissue, and 0.01% VK₂MS has already completely released at this time. Thus, it is preferable to use 0.1% VK₂MS or 1.0% VK₂MS, which have a longer release time. However, in the co-culture experiment of MG-63 cells and VK₂MS, it was observed that 1.0% VK₂MS has a more significant cell growth inhibition effect than that of 0.1% VK₂MS. Therefore, the desired effect can be achieved by using a smaller amount of 1.0% VK₂MS.

The present disclosure provides a delayed VK₂ drug release system, vitamin K₂ microsphere (VK₂MS). Compared to VK₂, VK₂MS is not only capable of decreasing the cell growth rate inhibition of VK₂, but also increasing the differentiation of cell ALP activity. Moreover, the diffusion drug release control technique of a polymer matrix was selected in this system to reduce the risk of requiring several surgeries. Furthermore, VK₂ can not only inhibit the activity of osteoclasts but also induce osteoblasts to differentiate to bone cells. Therefore, this technique has a very high value in future medical research and application. Combining the technique and tissue engineering scaffolds in vivo to treat osteoporosis or repair damaged bone tissue, and the expectation that it can be applied to bone tissue repair engineering would benefit all mankind.

### Other Embodiments

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and, without departing from the spirit and scope thereof, can make various changes and modifications of the invention to adapt it to various usages and conditions. As such, other embodiments are also within the claims.

What is claimed is:

1. A vitamin K₂ microsphere, comprising:
a particle formed of a poly(lactic-co-glycolic acid) (PLGA), and
vitamin K₂,
wherein
the PLGA has a molecular weight of 1000-300000 and contains lactic acid repeat units and glycolic acid repeat units, the molar ratio between the lactic acid repeat units and the glycolic acid repeat units being 1-9:9-1, and
vitamin K₂ is embedded in the particle and constitutes 0.005-75% by weight of the vitamin K₂ microsphere.

2. The vitamin K₂ microsphere of claim 1, wherein the microsphere has a particle size of 1-150 μm.

3. The vitamin K₂ microsphere of claim 1, wherein the microsphere contains vitamin K₂ in the amount of 0.01-0.3 mg.

4. The vitamin K₂ microsphere of claim 1, wherein the PLGA has a viscosity of 0.1-3 dl/g.

5. The vitamin K₂ microsphere of claim 2, wherein the microsphere contains vitamin K₂ in the amount of 0.01-0.3 mg.

6. The vitamin K₂ microsphere of claim 5, wherein the PLGA has a viscosity of 0.1-3 dl/g.

7. The vitamin K₂ microsphere of claim 2, wherein the PLGA has a viscosity of 0.1-3 dl/g.

8. The vitamin K₂ microsphere of claim 4, wherein the microsphere contains vitamin K₂ in the amount of 0.01-0.3 mg.

9. A method of preparing vitamin K₂ microspheres, the method comprising:
providing a vitamin K₂ solution that contains vitamin K₂, a polylactic-co-glycolic acid) (PLGA), and a first solvent;
providing a polyvinyl alcohol (PVA) solution that contains polyvinyl alcohol and a second solvent;
forming a vitamin K₂ emulsion by mixing the vitamin K₂ solution with the PVA solution; and
removing the first and second solvents to obtain vitamin K₂ microspheres, each of which contains the PLGA and vitamin K₂ embedded in a particle formed of the PLGA, wherein the first solvent is an organic solvent, the second solvent is water, the PLGA has a molecular weight of 1000-300000 and contains lactic acid repeat units and glycolic acid repeat units, the molar ratio between the lactic acid repeat units and the glycolic acid repeat units is 1-9:9-1; and vitamin K₂ constitutes 0.005-75% by weight of the vitamin K₂ microspheres.

10. The method of claim 9, wherein the method further comprising purifying the vitamin K₂ microspheres thus obtained via filtration or centrifugation.

11. The method of claim 9, wherein the weight/volume ratio between vitamin K₂ and the first solvent is 0.005-75%.

12. The method of claim 9, wherein the first solvent is dichloromethane, chloroform, tetrahydrofuran, dimethylformamide, benzene, toluene, or a combination thereof.

13. The method of claim 9, wherein a plasticizer is mixed with the vitamin K₂ solution and the PVA solution to form the vitamin K₂ emulsion.

14. The method of claim 13, wherein the the weight/volume ratio between vitamin K₂ and the first solvent is 0.005-75%

15. The method of claim 10, wherein the weight/volume ratio between vitamin K₂ and the first solvent is 0.005-75% and the first solvent is dichloromethane, chloroform, tetrahydrofuran, dimethylformamide, benzene, toluene, or a combination thereof.

16. The method of claim 15, wherein a plasticizer is mixed with the vitamin K₂ solution and the PVA solution to form the vitamin K₂ emulsion.

17. The method of claim 11, wherein the first solvent is dichloromethane, chloroform, tetrahydrofuran, dimethylformamide, benzene, toluene, or a combination thereof.

18. The method of claim 17, wherein a plasticizer is mixed with the vitamin K₂ solution and the PVA solution to form the vitamin K₂ emulsion.

19. A method of treating osteoporosis, the method comprising administering to a subject in need thereof an effective amount of the vitamin K₂ microsphere of claim 1.

20. A pharmaceutical composition comprising the vitamin K₂ microsphere of claim 1 and a pharmaceutically acceptable carrier.
